(19) United States
(12) Patent Application Publication
(10) Pub. No.: US 2008/0085881 A1
(43) Pub. Date: Apr. 10, 2008

(54) Stop-and-go oxaliplatin treatment regimens

(76) Inventor: Aimery De Gramont, Le Raincy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/543,940

(22) Filed: Oct. 6, 2006

(51) Int. Cl. A61K 31/555

(52) U.S. Cl. 514/185

(57) ABSTRACT

Methods of treating an oxaliplatin-sensitive cancer are disclosed in which a therapy comprising oxaliplatin is initiated, continued until a predetermined endpoint, stopped, then reintroduced after specific criteria are met. This stop-and-go approach reduces or eliminates the neurotoxicity associated with oxaliplatin administration while at the same increasing one or more of the beneficial effects of oxaliplatin therapy.

### FIELD OF THE INVENTION

This invention relates to methods of treating cancers, such as advanced colorectal cancer, by administering oxaliplatin in stop-and-go treatment regimens.

### BACKGROUND OF THE INVENTION

Oxaliplatin is an organoplatinum complex in which the platinum atom is complexed with 1,2-diaminocyclohexane and an oxalate ligand as a leaving group. It is believed to form inter- and intrastrand Pt-DNA adducts/cross-links that inhibit DNA replication and transcription.

Clinicians use oxaliplatin, either alone or in combination, in various dosages and administration protocols for the treatment of cancers. For example, oxaliplatin therapy is currently used as a second-line therapy to treat advanced colorectal cancer in patients that received a 5-fluorouracil (“5-FU”)-based first-line therapy. E.g. Maindrault-Goebel et al., Annals Oncol. (2004); 15:1210; Scartozzi et al., Oncol. (2005); 68:212. The American Cancer Institute estimates that approximately 150,000 people are diagnosed with colorectal cancer each year. In 2006, approximately 55,000 individuals will die from this cancer. It is the second leading cause of cancer deaths in Western countries.

Oxaliplatin can be used in combination with other chemotherapeutic agents. In particular, the combination of oxaliplatin with leucovorin and 5-FU is an effective treatment in previously treated patients with advanced colorectal cancer. This combination significantly improves response rates, extends progression-free and overall survival, and permits surgical resection in a subset of patients with previously non-resectable metastases. Oxaliplatin-based treatments can also be used to treat gastric cancer.

Unfortunately, oxaliplatin can result in sensory neurotoxicity, requiring treatment discontinuation. This neurotoxicity is reversible, however, suggesting that oxaliplatin could be reintroduced after the neurotoxicity subsided. There is accordingly a need in the art for oxaliplatin-based treatment regimens that increase one or more of the beneficial effects of oxaliplatin while reducing its neurotoxicity. It is, therefore, an object of the invention to provide new treatment regimens comprising oxaliplatin that meet this need.

In many embodiments of the invention, oxaliplatin treatment involves a “stop-and-go” regimen in which a therapy comprising oxaliplatin is initiated, continued until a predetermined endpoint, stopped, then reintroduced after specific criteria are met. In some embodiments, the oxaliplatin therapy is stopped after a defined period of time. A predetermined number of treatment cycles is used as the stopping point in other embodiments of the invention. In yet other embodiments, it is the development of a mild neuropathy that is the event that triggers the stop in oxaliplatin therapy. In some embodiments, the oxaliplatin therapy is begun, stopped, then reintroduced once, while in other embodiments this stop-and-go therapy is repeated more than once.

### SUMMARY OF THE INVENTION

The invention provides treatment regimens comprising oxaliplatin that increase one or more of the beneficial effects of this agent, reduce its neurotoxicity, or both increase one or more of its beneficial effects and reduce its neurotoxicity. In the various embodiments, this is achieved by providing “stop-and-go” regimens in which a therapy comprising oxaliplatin is initiated, continued until a predetermined endpoint, stopped, then reintroduced after specific criteria are met.

The stop-and-go therapies of the invention can provide beneficial effects, such as an increase in survival or a reduction in the cost of treatment. The stop-and-go therapies also may reduce or prevent the neuropathy often associated with continuous oxaliplatin-based therapies.

Thus, the invention provides the following embodiments:

In one embodiment, the invention provides a method of treating an oxaliplatin-sensitive cancer in a previously untreated patient, comprising:

a) administering from two to six cycles of a therapy comprising oxaliplatin to the patient;

b) stopping oxaliplatin treatment for a period of time during which the patient is maintained on 5-fluorouracil either alone or in combination with leucovorin; and

c) reintroducing treatment of the patient with a second therapy comprising oxaliplatin.

In some embodiments, the oxaliplatin-sensitive cancer is colorectal cancer.

In other embodiments, the oxaliplatin treatment is stopped for twelve cycles or until disease progression, whichever is earlier.

In still other embodiments, the first therapy and the second therapy comprising oxaliplatin each comprises oxaliplatin 130 mg/m² and leucovorin 400 mg/m² given concurrently as 2-hour infusions on day 1, followed directly by a 46 hour infusion of 5-FU at 2400 mg/m².

In yet other embodiments, the first therapy and the second therapy comprising oxaliplatin each comprises oxaliplatin 100 mg/m² and leucovorin 400 mg/M² given concurrently as 2-hour infusions on day 1, followed directly by a 46 hour infusion of 5-FU at 3000 mg/m².

In another embodiment, the invention provides a method of treating advanced colorectal cancer, comprising:

a) administering from two to six cycles of a first therapy comprising a 2-hour infusion of 100 mg/m² oxaliplatin to the patient having advanced colorectal cancer;

b) stopping oxaliplatin treatment for a period of time during which the patient is maintained on 5-fluorouracil either alone or in combination with leucovorin; and

c) reintroducing treatment of the patient with a second therapy comprising oxaliplatin.

In some embodiments, the patient with advanced colorectal cancer is a previously untreated patient.

In other embodiments, the oxaliplatin treatment is stopped for twelve cycles or until disease progression, whichever is earlier.

In still other embodiments, the first therapy and the second therapy each further comprise a 2-hour infusion of 400 mg/m² of leucovorin concurrent with the oxaliplatin infusion, followed immediately by a 46 hour continuous infusion of 5-fluorouracil at 2,400 mg/m².

In yet other embodiments, the first therapy and the second therapy each further comprise a 2-hour infusion of 400 mg/m² leucovorin concurrent with the oxaliplatin infusion, followed immediately by a 46 hour continuous infusion of 5-fluorouracil at 3,000 mg/m².

The invention also provides in one embodiment a method of treating advanced colorectal cancer in a previously untreated patient, comprising:

a) administering six cycles of a therapy comprising a 2-hour infusion of 100 mg/m² oxaliplatin to the patient;

b) stopping oxaliplatin treatment for a period of time; and

c) reinitiating treatment of the patient by administering an additional six cycles of a therapy comprising a 2-hour infusion of 100 mg/m² oxaliplatin to the patient.

In other embodiments, the oxaliplatin treatment is stopped for twelve cycles or until disease progression, whichever is earlier.

In still other embodiments, the therapy comprising oxaliplatin further comprise a 2-hour infusion of 400 mg/m² of leucovorin concurrent with the oxaliplatin infusion, followed immediately by 46 hours continuous infusion of 5-fluorouracil at 2,400 mg/m².

In yet other embodiments, the therapy comprising oxaliplatin further comprise a 2-hour infusion of 400 mg/m² leucovorin concurrent with the oxaliplatin infusion, followed immediately by a 46 hour continuous infusion of 5-fluorouracil at 3,000 mg/m².

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DESCRIPTION OF THE EMBODIMENTS

Chemotherapeutic agents, used either alone or in combination, improve overall survival rates and lengthen the time to progression in cancer patients. For many cancers, there are established clinical protocols that administer a particular chemotherapeutic agent, or combination of agents, at a specific dosage and in a particular delivery format. Although the quest for new agents continues, improvements in cancer patient survival and quality of life also come from new treatment protocols that improve the effectiveness, reduce the harmful side-effects, or both improve the effectiveness and reduce the harmful side-effects of, known agents. Thus, although a chemotherapeutic agent or a combination of chemotherapeutic agents may have been used previously to treat a particular cancer in a particular subset of patients, new dosing regimens and different administration sequences can provide novel treatment advantages and reduction in treatment costs.

Terms used to describe cancer therapy can have specialized meanings. It is therefore useful to provide the following definitions:

“5-FU” is an abbreviation for the agent 5-fluorouracil.

“Leucovorin” is also known as folinic acid.

“Regimen,” “treatment,” “therapy,” and the various combinations of and grammatical variations of these terms are used interchangeably in the description of the embodiments and elsewhere. These terms refer broadly to methods by which a disease such as a cancer is treated unless a different meaning is clearly indicated.

“Adjuvant” therapy is a therapy given in support of another treatment. For example, chemotherapy following surgical resection of a tumor aids in the elimination of any residual tumor cells, and therefore the chemotherapy is adjuvant therapy and supports the surgical treatment.

“FOLFOX” is any therapy in which the chemotherapeutic agents folinic acid [leucovorin], 5-fluorouracil, and oxaliplatin are administered in a treatment cycle. The dosage of each agent, the timing of the administration of each agent, and the form (e.g., bolus or continuous infusion) of the administration can vary. A FOLFOX therapy that includes a numerical designation (e.g., “FOLFOX4”) defines a specific combination of dosage, administration timing, and administration form.

A treatment “cycle” is the basic, repeated unit within a cancer treatment regimen. By way of example only, in the FOLFOX7 regimen one treatment “cycle” comprises oxaliplatin 130 mg/m² and leucovorin 400 mg/m² given concurrently as 2-hour infusions on day 1, followed directly by a 46 hour infusion of 5-FU at 2400 mg/m². If the I-leucovorin isomer is used, the dosage is 200 mg/m². This treatment cycle is generally repeated every 2 weeks. This example serves only to illustrate that a “cycle” involves administering one or more chemotherapeutic agents at a defined dose or dosages, in a defined form or forms (e.g., bolus injection or continuous infusion), and at a defined time or times. The example is not intended to limit a treatment “cycle” to the illustrated agents, dosages, timing of administration, or form of administration.

A therapy is designated a “first-line” therapy if it is the first therapy administered to a cancer patient following diagnosis. “Second-line” therapies are those initiated after the termination of a first-line therapy. Generally, a second-line therapy is initiated because the patient has become refractory to the first-line therapy.

“Reintroduction” refers to the re-initiation of a particular therapy after discontinuation. The reintroduced agent or agents may be used at the same or a different dosage as in a previous therapy, and may be administered using the same or a different timing and form as in a previous therapy.

A “stop-and-go” therapy is one that includes at least one planned withdrawal of one or more chemotherapeutic agents used in the therapy and a planned reintroduction of that chemotherapeutic agent or agents. A stop-and-go regimen may included planned withdrawal and reintroduction at specific end point. For example, the withdrawal and reintroduction can be based upon a certain number of cycles. The withdrawal may also be initiated when a certain side effect develops, and reintroduction made after the side effect diminishes or disappears.

Oxaliplatin is used in combination with leucovorin and 5-FU to treat advanced colorectal cancer and to treat stage III colon cancer following resection of the primary tumor. Oxaliplatin-based treatments can also be used to treat gastric cancer.

Treatment regimens using oxaliplatin with leucovorin and 5-FU are known as “FOLFOX” regimens (folinic acid [leucovorin], 5-fluorouracil, oxaliplatin), and different regimens are given different numerical designations to indicate differences in, among other things, the dosage of each drug used, the day(s) of administration, and the nature of the infusion. For example, the FOLFOX4 regimen comprises oxaliplatin 85 mg/m² given as a 2-hour infusion on day 1, leucovorin 200 mg/m² given as a 2-hour infusion on days 1 and 2, and 5-FU given on days 1 and 2 as a bolus of 400 mg/m² followed by a 22-hour infusion of 600 mg/m². The oxaliplatin infusion on day 1 is given concurrently with leucovorin, and on each day the 5-FU bolus follows the 2-hour infusion of leucovorin. This constitutes one “cycle,” and it is repeated every 2 weeks. FOLFOX7 is another example of a FOLFOX therapy. One cycle of FOLFOX7 comprises oxaliplatin 130 mg/m² and leucovorin 400 mg/m² given concurrently as 2-hour infusions on day 1, followed directly by a 46 hour infusion of 5-FU at 2,400 mg/m². FOLFOX7 is also repeated every 2 weeks. In the FOLFOX4 and FOLFOX7 regimens, the leucovorin dosages are given for the dI-leucovorin isomer. The I-leucovorin isomer can also be used, but in that case the mg/m² dosage is halved.

Although FOLFOX is used clinically to treat advanced colorectal cancer, patients treated with oxaliplatin can develop sensory neurotoxicity. This neuropathy is reversible, however, suggesting that oxaliplatin could be reintroduced after the neuropathy subsides. The development of sensory neuropathy is a significant problem because it often causes patients whose cancers are responding to the treatment to discontinue it.

Sensory neurotoxicity can be measured using known techniques and graded using defined criteria. For example, the National Cancer Institute Common Toxicity Criteria (NCI CTC) version 2.0 can be used to grade sensory neuropathy. Using these criteria, an NCI grade of 0 means that there is no change associated with the treatment. NCI grade 1 indicates the loss of deep tendon reflexes or paresthesia (including tingling) not interfering with function. NCI grade 2 indicates objective sensory loss or paresthesia (including tingling) that interferes with function but does not interfere with activities of daily living. NCI Grade 3 indicates sensory loss or paresthesia that interferes with activities of daily living. NCI Grade 4 is reserved for permanent sensory loss that interferes with function.

It is therefore an object of the invention to provide treatment regimens that reduce the sensory neurotoxicity associated with oxaliplatin administration. In some embodiments, patients receiving the treatment do not experience any sensory neuropathy. These patients have an NCI CTC score of 0. In other embodiments, one or more patient experiences some sensory neuropathy, but no patient has an NCI CTC score of greater than 2. In yet another embodiment, one or more patient experiences some sensory neuropathy, but no patient has an NCI CTC score of greater than 3.

In another embodiment of the invention, the treatment regimens avoid particular symptoms of neurotoxicity. For example, in certain treatment methods of the invention the patient receiving that treatment does not develop one or more of the following neurological symptoms: loss of deep tendon reflexes, deficit in proprioception, paresthesia, dysesthesia, hypoesthesia, dysarthria, Lhermitte's sign, cranial nerve palsies, and fasciculation.

In other embodiments of the invention, the treatment regimen increases one or more of the beneficial effects of oxaliplatin administration. Thus, in certain treatment methods the patient experiences prolonged disease-free survival, prolonged overall survival, a longer time to tumor progression, or a combination of one or more of these beneficial effects. The increase in the one or more beneficial effects can be measured by comparing the results for the patient receiving oxaliplatin to the results for another patient or cohort of patients given a treatment regimen that does not include oxaliplatin. For example, when the treatment regimen is a FOLFOX regimen, the increase in one or more beneficial effects can be determined by statistical comparison to a treatment regimen that administers the same number of cycles comprising leucovorin and 5-FU at the same dosages, timing, and form as in the FOLFOX regimen. Alternatively, an increase in one or more beneficial effects can be determined by statistical comparison of the same parameters (e.g., time to progression) for a treatment regimen that has Food and Drug Administration approval for the treatment of that particular type of cancer.

In at least some embodiments of the invention, both an increase in one or more of the beneficial effects of oxaliplatin and a reduction in oxaliplatin neurotoxicity are achieved.

Oxaliplatin is administered in a treatment cycle with one or more additional chemotherapeutic agents in many embodiments of the invention. For example, in some embodiments, the treatment cycle comprises a combination of oxaliplatin, leucovorin (folinic acid), and 5-FU. In other embodiments, the treatment cycle comprises oxaliplatin and irinotecan, with or without 5-FU. In still other embodiments, however, oxaliplatin is the only chemotherapeutic agent administered during the treatment cycle.

The oxaliplatin used in the methods of the invention can be administered in different dosages, timings, and forms. For example, in some embodiments of the invention, oxaliplatin is used at a dosage of 130 mg/m² given as a 2-hour infusion on day 1 of the treatment cycle. Oxaliplatin is used in other embodiments at a dosage of 100 mg/m² given as a 2-hour infusion on day 1 of the treatment cycle. For example, oxaliplatin 100 mg/m² can be used with a 5-FU infusion of 3,000 mg/m². Other dosages of oxaliplatin are also possible, including 75 mg/m² and 65 mg/m². Of course, all numerical values for dosages are approximate and variations in the actual dosage given are still considered a dosage meeting the specific value. Ultimately, the dosage given to a particular patient will be determined by his clinician and will be based upon those factors unique to that patient. The dosage may also vary depending upon the type of cancer to be treated and whether oxaliplatin is used alone or in combination with other agents. In general, however, oxaliplatin is administered in the methods of the invention in a range of 65 mg/m² to 130 mg/m².

Examples of therapies comprising oxaliplatin include the FOLFOX regimens. The FOLFOX4 regimen comprises oxaliplatin 85 mg/m² given as a 2-hour infusion on day 1, leucovorin 200 mg/m² given as a 2-hour infusion on days 1 and 2, and 5-FU bolus of 400 mg/m² given on days 1 and 2, followed directly by a 22-hour infusion of 5-FU 600 mg/m² on each day. The oxaliplatin infusion on day 1 is given concurrently with leucovorin, and on each day the 5-FU bolus directly follows the 2-hour infusion of leucovorin. FOLFOX7 comprises oxaliplatin 130 mg/m² and leucovorin 400 mg/m² given concurrently as 2-hour infusions on day 1, followed directly by a 46 hour infusion of 5-FU at 2,400 mg/m². Both the FOLFOX4 and the FOLFOX7 cycles are two weeks in duration. In both regimens, the leucovorin dosages are for the dI-leucovorin isomer. The I-leucovorin isomer can also be used, but in that case the mg/m² dosage is halved.

Stop-and-go therapies can take various forms, but in each case the therapy comprising oxaliplatin is initiated, continued until a predetermined endpoint, stopped, then reintroduced after specific criteria are met. The predetermined endpoint can be a certain number of treatment cycles, a certain period of time, or the development of a measurable level of neuropathy. Similarly, the specific criteria that trigger reintroduction of oxaliplatin therapy can be a certain of period of time, a certain number of treatment cycles using chemotherapeutic agents other than oxaliplatin, the diminishment or disappearance of symptoms of neuropathy, or a combination of these criteria.

For example, in one stop-and-go therapy, a patient receives FOLFOX7 for 6 cycles. FOLFOX7 is then stopped and the patient is maintained on a combination of leucovorin and 5-FU (“LV5FU”), followed by reintroduction of FOLFOX7 for 6 cycles. In this approach, both the “stop” and the “go” is predetermined: the “stop” occurs after a fixed number of cycles and the “go” occurs after a predetermined period of maintenance therapy. The number of cycles of LV5FU between the “stop” and the “go” can vary, but can be, for example, 12 cycles, 11 cycles, 10 cycles, 9 cycles, 8 cycles, 7 cycles, 6 cycles, 5 cycles, 4 cycles, 3 cycles, 2 cycles, or 1 cycle.

Stop-and-go therapy can be used to treat different cancers, such as advanced colorectal cancer or gastric cancer. For example, advanced colorectal cancer can be treated using 6 cycles of FOLFOX7, 12 cycles of LV5FU maintenance therapy during the “stop” period, followed by 6 additional cycles of FOLFOX7 during the “go” period. In some embodiments, the advanced colorectal patient treated with the stop-and-go therapy is an untreated patient—that is, the patient has not received any chemotherapy to treat the colorectal cancer prior to beginning the stop-and-go therapy. In such patients, the stop-and-go therapy is a first-line therapy.

In still other embodiments of the invention, the stop-and-go therapy comprises a modified FOLFOX7 therapy. Modified FOLFOX7 is the same as FOLFOX7 except that the dose of oxaliplatin is decreased from 130 mg/m² to 100 mg/m² and the 5-FU infusion is increased to 3,000 mg/m². That is, a modified FOLFOX7 comprises oxaliplatin 100 mg/m² and leucovorin 400 mg/m² given concurrently as 2-hour infusions on day 1, followed directly by a 46 hour infusion of 5-FU at 3,000 mg/m². If the I-leucovorin isomer is used, the mg/m² dosage of leucovorin is halved.

### EXAMPLE 1

Stop-And-Go Therapy in Patients with Advanced Colorectal Cancer.

The OPTIMOX1 study examined the effect of oxaliplatin reintroduction in previously untreated patients with advanced colorectal cancer. The details of this study have been recently published by Tournigand et al., J. Clin. Oncol. (January 2006);24:394, and they are incorporated herein by reference. Two different FOLFOX regimens were used in the study: FOLFOX4 and FOLFOX7. The FOLFOX4 regimen comprises oxaliplatin 85 mg/m² given as a 2-hour infusion on day 1, leucovorin 200 mg/m² given as a 2-hour infusion on days 1 and 2, and 5-FU given on days 1 and 2 as a bolus of 400 mg/m² that is followed directly by a 22-hour infusion of 5-FU 600 mg/m² on each day. The oxaliplatin infusion on day 1 is given concurrently with leucovorin, and on each day the 5-FU bolus directly follows the 2-hour infusion of leucovorin. FOLFOX7 comprises oxaliplatin 130 mg/m² and leucovorin 200 mg/m² given concurrently as 2-hour infusions on day 1, followed directly by a 46 hour infusion of 5-FU at 2400 mg/m². In some patients, the I-leucovorin isomer was used, but at half the mg/m² dosage for dI-leucovorin.

Patients were randomized to either FOLFOX4 every 2 weeks until progression (arm A), or FOLFOX7 for 6 cycles, maintenance with leucovorin-5-FU but without oxaliplatin for 12 weeks, and reintroduction of FOLFOX7 (arm B). There was no difference between arms in response rate, progression-free survival, and overall survival. It was not possible to determine whether oxaliplatin reintroduction had an effect on survival by directly comparing the two arms of the OPTIMOX1 study, because a significant number of patients in both arms of the OPTIMOX1 study received oxaliplatin after being taken off the study. Oxaliplatin reintroduction was, however, identified as an independent prognostic factor for improved overall survival. Also, stopping oxaliplatin after the sixth cycle was associated with a reduced risk of grade 3 or 4 neurotoxicity.

A retrospective analysis was used to evaluate the effect of oxaliplatin reintroduction on overall survival. The percentage of patients with oxaliplatin reintroduction was calculated in each center regardless of treatment arm, number of patients, or whether reintroduction was per protocol. A Cox model was used to analyze the impact of oxaliplatin reintroduction on survival, after adjustment for all baseline covariates, and using random effects to account for the fact that the percentage of patients with oxaliplatin reintroduction was estimated in each center. Centers were grouped by percentage of oxaliplatin reintroduction into 4 reintroduction classes defined according to the percentage of patients with oxaliplatin reintroduction (0, 1-20, 21-40, and >40). The hazard ratio (HR; adjusted for all baseline covariates) was calculated for each reintroduction class, using centers with no reintroduction (0%) as the reference group.

After adjustment for all other factors, the percentage of patients with oxaliplatin reintroduction had a highly significant impact on survival (p<0.001). Reintroduction class also had a significant impact on survival, after adjustment for all other factors (p=0.02; Table below). Median survival increased from 15 months in the reference group (0% reintroduction) to 23 months in the >40% reintroduction class.

TABLE 1

| % Patients with | Mean % with | | Total | Median | Adjusted survival |
|---|---|---|---|---|---|
| oxaliplatin | oxaliplatin | | no. of | treatment | hazard ratio (95% |
| reintroduction | reintroduction | No. of centers | patients | lines | C.I.) |
| 0 | 0 | 18 | 37 | 3 | 1.00 (reference) |
| 1–20 | 15 | 6 | 100 | 3 | 0.91 (0.56–1.48) |
| 21–40 | 34 | 12 | 227 | 3 | 0.78 (0.50–1.21) |
| >40 | 55 | 21 | 257 | 3 | 0.59 (0.38–0.91) |

This retrospective analysis indicates that oxaliplatin reintroduction has a significant impact on survival in patients with advanced colorectal cancer.

A cost-minimization analysis based on the efficacy results of the OPTIMOX1 study was also conducted. The analysis was performed from the perspective of the third party payer and included only direct medical costs: chemotherapy, hospitalization and management of side effects. The horizon time was from inclusion until patient death. Sensitivity analyses were performed on drug costs and full/day hospitalization rates.

Hospitalization costs per patient were the main driver cost. Hospitalization represented [symbol] 6,595 in the FOLFOX7 arm versus [symbol] 10,522 in the FOLFOX4 arm reflecting the decrease of number of hospitalization days (p<10⁻³). Chemotherapy costs per patient were comparable in each arm of treatment despite higher doses of oxaliplatin with FOLFOX7 ( [symbol] 6,870) compared to FOLFOX4 ( [symbol] 7,047) (p=0.30). The management of side effects in both groups appeared very low in cost, compared to hospitalization and drug costs with [symbol] 271 and [symbol] 382 (p=0.11) for FOLFOX4 and FOLFOX7 respectively. The mean total cost per patient was higher in FOLFOX4 arm than in FOLFOX7 arm with [symbol] 17,841 versus [symbol] 13,847 respectively (p<10⁻³). The FOLFOX7 regimen with intermittent oxaliplatin treatment (stop-and-go) is therefore cost saving compared with FOLFOX4 regimen.

Thus, the stop-and-go methods comprising oxaliplatin therapy are not only beneficial to the patient because they reduce or prevent oxaliplatin-associated neuropathy, but they are also beneficial to public policy because they are more economical.

Although certain embodiments have been described in detail, the embodiments within the specification provide only an illustration of embodiments of the invention and should not be construed to limit the scope of the invention. The skilled artisan readily recognizes that many other embodiments are encompassed by the invention. The invention is most thoroughly understood in light of the teachings of the references cited within the specification. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Also, it is important to note that, unless otherwise indicated, all numbers used in the specification, including in the claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Similarly, the term “at least” preceding a series of elements is to be understood to refer to every element in the series, unless otherwise indicated.

Other embodiments of the invention will be apparent to those skilled in the art from consideration of the specification and practice of the invention disclosed herein. The described embodiments are to be considered in all respects as illustrative only and not restrictive. The scope of the invention is, therefore, indicated by the appended claims rather than by the foregoing description. All changes which come within the meaning and range of equivalency of the claims are to be embraced within their scope.

What is claimed is:

1. A method of treating an oxaliplatin-sensitive cancer in a previously untreated patient, comprising:
a) administering from two to six cycles of a therapy comprising oxaliplatin to the patient;
b) stopping oxaliplatin treatment for a period of time during which the patient is maintained on 5-fluorouracil either alone or in combination with leucovorin; and
c) reintroducing treatment of the patient with a second therapy comprising oxaliplatin.

2. The method of claim 1, wherein the oxaliplatin-sensitive cancer is colorectal cancer.

3. The method of claim 1, wherein the oxaliplatin treatment is stopped for twelve cycles or until disease progression, whichever is earlier.

4. The method of claim 1, wherein the first therapy and the second therapy comprising oxaliplatin each comprises oxaliplatin 130 mg/m² and leucovorin 400 mg/m² given concurrently as 2-hour infusions on day 1, followed directly by a 46 hour infusion of 5-FU at 2400 mg/m².

5. The method of claim 1, wherein the first therapy and the second therapy comprising oxaliplatin each comprises oxaliplatin 100 mg/m² and leucovorin 400 mg/m² given concurrently as 2-hour infusions on day 1, followed directly by a 46 hour infusion of 5-FU at 3000 mg/m².

6. A method of treating advanced colorectal cancer, comprising:
a) administering from two to six cycles of a first therapy comprising a 2-hour infusion of 100 mg/m² oxaliplatin to the patient having advanced colorectal cancer;
b) stopping oxaliplatin treatment for a period of time during which the patient is maintained on 5-fluorouracil either alone or in combination with leucovorin; and
c) reintroducing treatment of the patient with a second therapy comprising oxaliplatin.

7. The method of claim 6, wherein the patient with advanced colorectal cancer is a previously untreated patient.

8. The method of claim 6, wherein the oxaliplatin treatment is stopped for twelve cycles or until disease progression, whichever is earlier.

9. The method of claim 6, wherein the first therapy and the second therapy each further comprise a 2-hour infusion of 400 mg/m² of leucovorin concurrent with the oxaliplatin infusion, followed immediately by a 46 hour continuous infusion of 5-fluorouracil at 2,400 mg/m².

10. The method of claim 6, wherein the first therapy and the second therapy each further comprise a 2-hour infusion of 400 mg/m² leucovorin concurrent with the oxaliplatin infusion, followed immediately by a 46 hour continuous infusion of 5-fluorouracil at 3,000 mg/m².

11. A method of treating advanced colorectal cancer in a previously untreated patient, comprising:
a) administering six cycles of a therapy comprising a 2-hour infusion of 100 mg/m² oxaliplatin to the patient;
b) stopping oxaliplatin treatment for a period of time; and
c) reinitiating treatment of the patient by administering an additional six cycles of a therapy comprising a 2-hour infusion of 100 mg/m² oxaliplatin to the patient.

12. The method of claim 11, wherein the oxaliplatin treatment is stopped for twelve cycles or until disease progression, whichever is earlier.

13. The method of claim 11, wherein the therapy comprising oxaliplatin further comprise a 2-hour infusion of 400 mg/m² of leucovorin concurrent with the oxaliplatin infusion, followed immediately by 46 hours continuous infusion of 5-fluorouracil at 2,400 mg/m².

14. The method of claim 11, wherein the therapy comprising oxaliplatin further comprise a 2-hour infusion of 400 mg/m² leucovorin concurrent with the oxaliplatin infusion, followed immediately by a 46 hour continuous infusion of 5-fluorouracil at 3,000 mg/m².
